(19) United States
(12) Patent Application Publication
(10) Pub. No.: US 2013/0231720 A1
(43) Pub. Date: Sep. 5, 2013

(54) METHOD AND APPARATUS FOR IRRADIATION OF IRREGULARLY SHAPED SURFACES

(71) Applicant: Friedrich Luellau, Adendorf (DE)

(72) Inventor: Friedrich Luellau, Adendorf (DE)

(73) Assignee: LUELLAU ENGINEERING GMBH, Lueneburg (DE)

(21) Appl. No.: 13/885,234

(22) PCT Filed: Oct. 11, 2011
(86) PCT No.: PCT/EP11/05099

§ 371 Date: May 14, 2013

(30) Foreign Application Priority Data
Nov. 15, 2010 (DE) 10 2010 051 162.5

(51) Int. Cl. A61N 5/06

(52) U.S. Cl. CPC A61N 5/0613
USPC 607/88

(57) ABSTRACT

The invention relates to a method for irradiating or treating areas of the body with electromagnetic radiation from a radiation source, the area of the body including at least one irregularly outlined treatment area, which is determined and irradiated. The invention is characterized in that the area of the body is divided up into a number of sub-areas, which at least partly contain the treatment area, and a proportion of the treatment area contained in each sub-area is irradiated with a radiation dose sequentially or in a scrolled manner or step-by-step or in a targeted manner.

The invention relates to a method for irradiation or treatment of body surfaces with electromagnetic irradiation from a radiation source, wherein the body surface contains at least one treatment surface with irregular edges, which surface is determined and irradiated.

Furthermore, the invention relates to an apparatus, for the apparatus for irradiation or treatment of surfaces, comprising at least one radiation source, at least one treatment head having optics for imaging a light modulator on a body surface, means for recognition of at least one treatment surface on the body surface to be irradiated, at least one light modulator, particularly a micromirror actuator.

The U.S. patent applications US A1 2003/0045916, US 2008/0051773A1, as well as DE T2 698 827 disclosed methods and systems for treatment of inflammatory, proliferative skin problems, such as, for example, psoriasis, using ultraviolet phototherapy. The methods and systems use optical techniques to scan the skin of a patient, to identify regions of affected skin, and to selectively administer high doses of phototherapeutic ultraviolet radiation to the identified regions.

In selective phototherapy, the demand for the greatest possible power in the spectral. UVA and UVB range is also technically problematical. For phototherapy in the UVA range, doses of up to 130 J/cm² are required on the skin surfaces, on the basis of medical guidelines. For this reason, among others, what is called PUVA therapy has been developed, in which the skin is clearly made more photosensitive by means of biochemical substances (for example psoralen). This means that after oral administration or topical treatment of the skin (cream or bath), the skin clearly becomes more sensitive to UV radiation. In PUVA therapy, irradiation doses of 0.1 J/cm² to as much as 10 J/cm² are required. In phototherapy with UVB radiation, lower doses are required, but they still lie in ranges from 0.05 J/cm² to approximately 1.0 J/cm².

The levels of the higher doses are typically greater than two minimal erythema doses (MED) and frequently about 10 MED. These dose levels are very effective in treatment of affected skin regions, but would severely damage non-affected skin regions, for example normal skin. In order to guarantee that only skin regions affected by psoriasis or another disorder are identified for the high UV radiation doses, the methods and systems use one or more optical diagnostics that relate to independent physiological characteristics of the affected skin.

In this connection, laser sources primarily serve as radiation sources; these irradiate the irregularly shaped treatment surfaces row by row by way of mirrors. It is true that such radiation sources are very powerful, but they have the serious disadvantage that they are also very expensive.

This disadvantage is avoided by the apparatus described in DE A1 10 2005 010 723.0, from which the present invention also proceeds, because there, a UV lamp is proposed as a radiation source, the light of which is directed at the body surface by means of a micromirror actuator, also known as a Digital Mirror Device. Micromirror actuators are micromechanical modules. They guide light in targeted manner, using mirrors that can be moved individually, so that the light, by means of a matrix-shaped arrangement, is projected to produce an image that is composed of the switched pixels of each mirror, by switching the individual mirrors. Synonyms, trademarks, and trade names of known manufacturers based on this technology are, among others, Digital Micromirror Device, DMD, from Texas Instruments, or Digital Light Processing (DLP).

These light modulators, or also light modulators that work by means of switchable liquid crystals, called LCDs, are connected with the imaging optics in a treatment head, together with a radiation source, to form a module that is used for directed emission of radiation in the direction of a target, for imaging the light modulator on a body surface. Consequently, a treatment head is a device for targeted, focused, and adapted application of radiation doses to an irregularly edged treatment surface that is part of a body surface.

However, with the UV lamps, which are significantly more cost-advantageous as compared with laser light sources, one also has to accept the disadvantage that the treatment time for the patient becomes longer, because only part of the light power emitted by the UV lamp can be captured by the optics and utilized optically.

There is therefore an urgent need for irradiation devices for inexpensive irradiation of selected skin surfaces, which furthermore also allow shorter therapy times.

It is the task of the invention to shorten the treatment time in the irradiation of patients.

In the case of a method for irradiation or treatment of body surfaces with electromagnetic radiation from a radiation source, whereby the body surface contains at least one irregularly edged treatment surface that is determined and irradiated, the task is accomplished in that the body surface, for example 630×840 mm, is divided up into a number of surface sections, e.g. 7×7 surface sections having a size of 90×120 mm, which contain the treatment surface, at least in part, and that a treatment surface portion contained in each surface section is exposed to light with a radiation dose sequentially or in scrolling or step-by-step or targeted manner. In this connection, radiation dose is understood to mean the product of time multiplied by the power of the radiation that impacts the treatment surface. At the same power of the UV lamp, the radiation is directed not at the entire body surface, but rather only onto the much smaller surface section. The power density, i.e. the power per surface area unit, is thereby increased by a factor, for example of 49. Accordingly, the irradiation time of the surface section can be reduced reciprocally, in order to allow the same dose to impact the irradiated surface section. If all the surface sections are irradiated one after the other, no reduction in the treatment time of the patient can be found. However, the disease profiles of a patient consist of treatment surfaces that cover the entire body surface only in extremely rare cases. Much more frequently, multiple smaller treatment surfaces having irregular edges are present on the body surface. In these cases, a great number of surface sections do not contain any treatment surface components at all, so that they do not require any irradiation. These surface sections can then be left out. Since only a subordinate amount of surface sections, generally 1 to 5 surface sections of a body surface need to be irradiated, the treatment times for the patient are reduced in surprisingly dramatic manner. The task of reducing the treatment time for the patient has thereby been accomplished.

Therefore, the power density is advantageously increased. In comparison with the body surface, in other words the maximal total irradiation surface, of 630×840 mm, for example, the surface section demonstrates an energy density that is 49 times as great. The total power available from the radiation source is not distributed over the available body surface in its entirety, but is only applied to selected surface sections. By means of the irradiation of selected surface sections, a time advantage occurs as compared with conventional methods, so that the economic efficiency of the method is also increased. With the reduction in the surface area of a surface section, the starting power of the radiation source can also be reduced, in some cases, so that lower acquisition costs occur and longer useful lifetimes for the light source are obtained. Shortened treatment times allow greater capacity utilization of the equipment, and therefore shortening of treatment times and waiting times. The irradiation dose advantageously lies between 0.05 J/cm² and 1.5 J/cm². One surface section is then irradiated sequentially after the preceding one, without any gaps, so that a mosaic-like image of the treatment surface is obtained. The surface sections of the body surface to be irradiated can be reached and exposed to light by means of row-by-row or column-by-column approach to the surface sections on the body surface. In the case of such a step-and-repeat method, the treatment head has to be constantly accelerated, moved, and braked in order to join the surface sections together again by means of a mechanical system.

However, instead of the step-and-repeat method just described, a direct approach to the surface sections to be exposed to light is better, because fewer acceleration and braking times occur.

The entire mechanics of the irradiation device are freed from the acceleration and braking forces that occur, to a great extent, if the surface sections that lie next to one another are approached in scrolling manner. In this connection, the are transferred to the row or column that lies next to them, in the manner of a rolling image of the row or column content, synchronous to the travel speed, and only the outermost column or row is newly written or deleted. Corresponding shift registers serve as memory.

[A2] In an embodiment of the method, it is provided that a topology of the treatment surface is determined. Because the treatment surface is not level, in many cases, but rather has higher and lower regions, i.e. the surface sections have normal lines that are oriented differently from the optical axis of the optics, corresponding power density differences also occur of the radiation that impacts a surface unit. After determination of the topology of the treatment surface, the influence can be calculated and a correction can take place for every pixel of the surface section, so that a dose corresponding to the damage can be administered independent of the angle position relative to the optical axis.

[A3] Because a radiation dose distribution for treatment of the treatment surface is determined, a corresponding individual radiation dose can reach the treatment surface as a function of the intensity of the damage, for each pixel of a surface section. Limit values that are set can also be defined in position-dependent manner, and can be adhered to precisely.

[A4] Because the treatment surface is repeatedly determined, position changes can be recognized quickly, as a function of the repetition frequency, and balanced out correspondingly. For example, after every irradiation of a surface section, the treatment surface can be determined anew, and the position of the surface sections can be corrected by the change vector that has been determined. The more frequently this happens, the more precisely and quickly the correction can take place. The limits are determined by the speed of the calculations. The calculation procedures required for this are not supposed to distort the irradiation of the surface sections.

[A5] If a maximal radiation power distribution on the surface section and/or body surface is also determined, light intensity errors of the imaging optics can advantageously also be compensated. To measure the radiation power distribution, all the surface sections of the body surface are impacted with non-modulated light, for example, and their local power is measured in suitable manner. In an ideal case, no local differences can be determined. This then also holds true for the pixels of a surface section. If, however, there are power differences of the pixels of a surface section, for example from the optical axis toward the edge, then the controller can undertake a corresponding correction, so that imaging errors do not impair the local dosage of the radiation.

[A6] Because the one radiation power distribution is adapted to the local radiation dose distribution by means of modulation of a light modulator, preferably a micromirror actuator, the radiation dose can be adjusted with particular precision to individually adapted treatment of diseased skin locations. The risk of overdose is minimized. By means of time-dependent intensity-modulated irradiation, it is advantageously possible to have a further positive influence on the skin surface, by means of a special time sequence of the irradiation dose.

[A7] If the radiation power density is adjusted by means of a change in an imaging scale of the light modulator, and if an image of the light modulator on the body surface is selected as a surface section, the maximal radiation density that can be achieved can also be adjusted with a micromirror actuator, in particularly elegant manner. Micromirror actuators are available in different sizes, shapes, and variants. It is advantageously possible to achieve therapeutically desirable threshold values in any desired manner, or to safely not exceed therapeutically dangerous limit values.

[A8] The task is also accomplished by an apparatus for irradiation or treatment of surfaces, comprising at least one radiation source, at least one treatment head having optics for imaging a light modulator on a body surface, means for recognition of at least one treatment surface in the body surface to be irradiated, at least one light modulator, particularly a micromirror actuator, in that the apparatus has a controller that is configured to divide the body surface up into surface sections, and has a position drive controlled by it, which drive is configured to direct the treatment head at the surface section as a function of the surface section to be irradiated. It is practical if the expanse of the irradiation surface is divided up into surface sections of the body surface. If such surface sections contain only a portion of the irradiation surface, this means that a section of the irregular edge of the irradiation surface passes through the surface section. Then, the pixels on the side of the irradiation surface are turned on for exposure to light, and the others are turned off, using a micromirror actuator. The pixels of surface sections without any section of the edge, but with an irradiation surface component, are completely turned on during exposure to light. The remaining surface sections, i.e. those without an edge and without an irradiation surface component, are not approached and not exposed to light. As a result, the treatment times are advantageously shortened.

[A9] In a preferred embodiment of the apparatus, it has a table for a body to lie on and a portal for movable fastening of the treatment head. A patient can assume a comfortable lying position on the table, and can relax. The treatment head can be freely disposed on the portal, above the patient. There, it can be freely positioned in multiple axes. By means of the free positioning of the treatment head, optimal irradiation of all skin surfaces is made possible. Advantageously, irradiation of curved skin surfaces is also possible in this manner, particularly if the supports of the portal are configured to pivot. Free positioning of the irradiation head additionally allows adjustment of the distance between treatment head and skin surface.

A10] The measure that the portal is configured to be displaceable relative to the table makes it possible to freely reach almost all skin locations of a patient, without having to undertake a change in position of the patient.

The method and the apparatus according to the invention can be commercially utilized for cosmetic administration of radiation, for example for tanning the skin. However, exposure of other biological substrates to light is also possible, within the scope of diagnostics and research. The irradiation device can, however, also find use in other industrial application sectors, such as, for example, photochemistry, photobiology, or UV adhesives technology, if the matter of concern is irradiation in the wavelength ranges from 280 nm to 2500 nm, with local precision and intensity modulation, for example for exposure of liquid plastics to light and their crosslinking, for the production of three-dimensional bodies.

A preferred embodiment of the invention will be explained as an example, using a drawing. The figures of the drawing show, in detail:

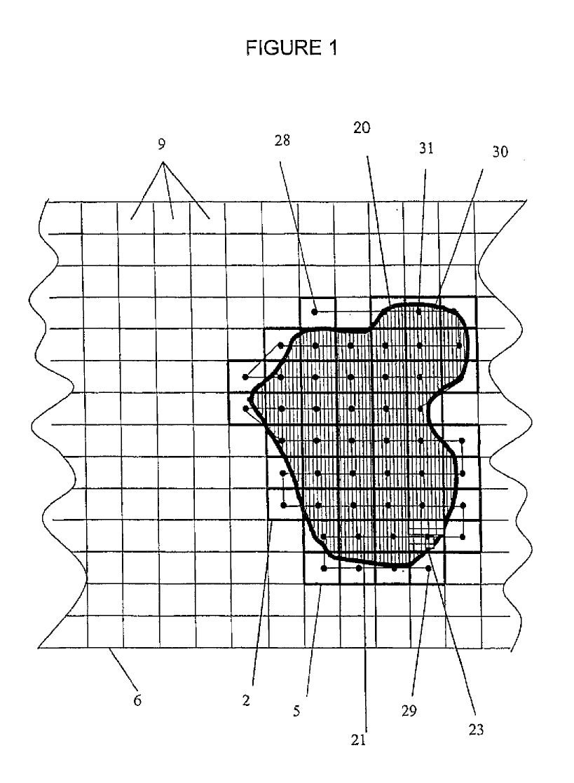

FIG. 1 a schematic representation of a treatment sequence,

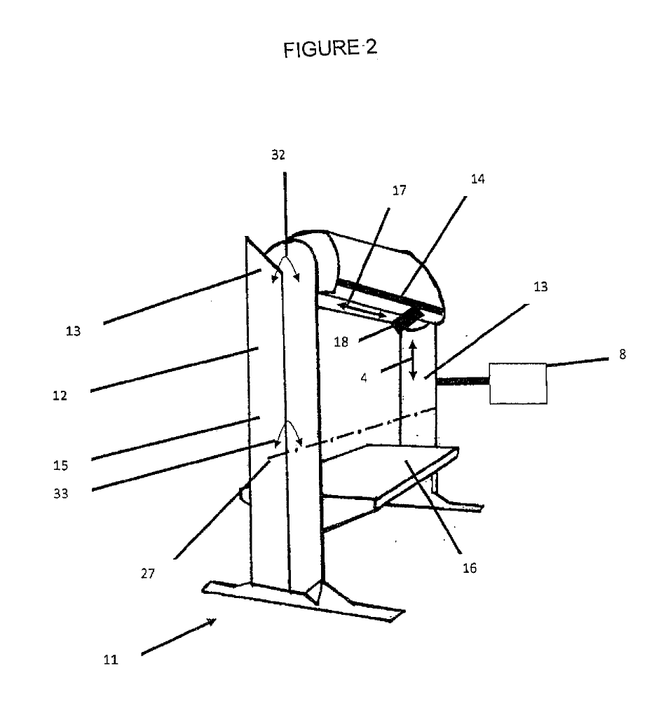

FIG. 2 a perspective view of the apparatus according to the invention, and

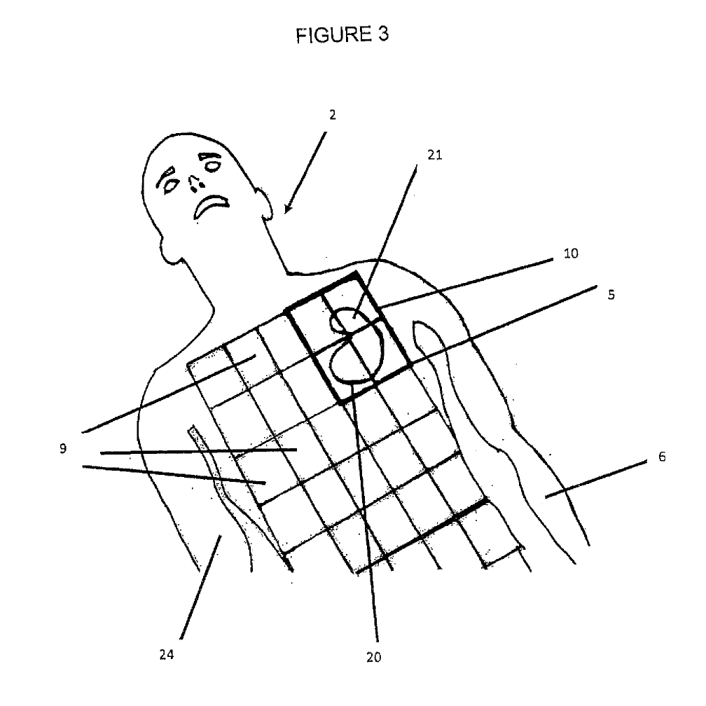

FIG. 3 a perspective view of a person lying on the lying surface of the apparatus, with a light grid projected onto the skin surface.

In FIG. 1, the maximal possible irradiation surface is shown, which is referred to in this application as the body surface 6. The body surface 6 characterizes the work region on the skin surface of a person 2 to be treated (FIG. 3), which can be reached by the treatment head 7, for example 70 cm×90 cm, in other words 6300 cm² in total for a one side or half of a human body. A treatment surface 20 is either determined by hand and entered into the controller 8, or recognized by means of automatic image recognition of damaged skin regions. To mark the body surface 6, a light frame 10 (FIG. 3) is projected onto the body surface 6. In this connection, it is advantageous if the grid of the light frame corresponds to the division of the body surface 6 into its matrix of surface sections, in the present case therefore 11×14 surface sections.

Image recognition is performed with a camera that also evaluates the projected grid or a projected stripe pattern for determining a topology of the treatment surface. For each surface section 9 or, even better, for each pixel of a surface section, its direction relative to the optical axis is determined and a correction factor is calculated, with which the radiation power is corrected, precisely by pixels, in such a manner that the desired dose is applied to each surface part.

Parameterization of the treatment surface 6, precisely defined by pixels, is input by the treatment personnel by means of a controller 8 (FIG. 2), or confirmed by automatic image recognition as a function of values proposed by a diagnosis that was made automatically and/or topology that was determined automatically and/or device-specific power distribution. In this connection, parameterization is dependent, among other things on the distribution and intensity of the diseased skin regions 21. On the basis of this parameterization, the treatment surface 6 is broken down, by the controller 8, into a group of surface sections 5 that contain portions of the treatment surfaces 21 and therefore have to be irradiated. The controller 8 is programmed in such a manner that if the parameterization is not input, grouping of all the surface sections of a body surface 6 is generated automatically.

The surface area total of all the surface sections 9 corresponds, in this connection, to the body surface 6. The individual surface section 9 corresponds to the imaged surface of the light modulator, preferably of the DMD. This DMD consists of a matrix of mirrors disposed in rows and columns, of which each represents a pixel 23 of the surface section 9.

Automatic image recognition is used to determine diseased skin regions 21 and their pixel-precise position. For this purpose, the treatment head 7 is moved over the entire body surface 6. Subsequently, a radiation spectrum suitable for image recognition and diagnosis is emitted onto the body surface by the treatment head 7. The reflections of the spectrum from the skin surface 23 are received by a camera. The disturbed, diseased skin regions 21 are diagnosed by means of analysis of the reflected and recorded radiation spectrum, and the diagnosis is assigned to each pixel. The resolution of the camera should therefore at least correspond to the number of pixels present on the body surface 6. If the resolution of the camera does not meet this requirement, diagnosis can also take place individually for each surface section 9 and be stored in the memory of the controller 8. In this case, a resolution of the camera that meets the pixel count of the light modulator would suffice.

Only the group 5 of the surface sections 9 that contain portions of treatment surfaces 21 are irradiated. Within the surface sections 9, in turn, only the surfaces of pixels 23 for which a corresponding diagnosis is available and that are therefore assigned to the treatment surface 21. A radiation sequence is shown in FIG. 1 in this regard. The group 5 of the surface sections 9 to be irradiated is irradiated sequentially, starting with the starting surface 28, in accordance with the sequence 30. In the case shown, the surface sections to be irradiated are approached row by row. From the starting surface 28, the treatment head 7 moves to the next stopping point 31 located to the right, and irradiates the related surface section 9. The surface section 9 that lies in between was skipped, in this connection, since it does not have any portion of treatment surfaces 21. By means of repeated step and repeat, the entire treatment surface 21 is irradiated along the sequence 30, which also represents the path of the treatment head 7, until the treatment is completed when the ending surface 29 has been reached. The dose of the radiation to be administered is stored in the memory of the controller 8 for every pixel 23. The maximal dose is the product of the maximal power with reference to the pixel surface multiplied by the irradiation period. The irradiation period is the same for all the surface sections 9. The power of the radiation that impacts the pixel surface of the treatment surface 21 is adjusted between zero and the maximal power by means of closing and opening of micromirrors at a variable scanning ratio, which takes place at high frequency. This frequency of the opening and closing of micromirrors thereby also changes the irradiation dose on the skin surface 24 during the treatment period. Influences of the optics on the power distribution in the surface section 9 and influences of the topology of the treatment surface 21 are corrected in the calculation of the dose, in such a manner that each surface section 9 receives the desired dose.

Image recognition of the treatment surface 21 is performed repeatedly. Position changes of the treatment surface 21 are recognized by means of a comparison of two image recognition results, and a vector of these changes is determined. The matrix of the pixels 23 to be irradiated is regularly corrected with this vector, so that movements of the patient during the treatment do not have any influence on the treatment.

In FIG. 2, an apparatus 11 for performing the method according to the invention is shown. The apparatus 11 consists of a frame, portal 12, the two side supports 13, and an upper, connecting cross-beam 14, and a table 16 that acts as a lower cross-beam comprises. The side supports 13 of the portal 12 are divided by an articulation 15, in each instance. By means of these articulations, it is possible to pivot the upper part of the portal by approximately 30 degrees relative to the lower part of the side supports 13, to each side. Below the articulations 15, a table 16 is provided, which serves as a lying surface for a patient 2. This table is connected with the side supports 13 in height-adjustable manner, to allow persons 2 to lie on it. The table 16 forms the lower cross-beam 14 of the frame 12. A linear drive 18 for displacement of the treatment head 7 along a horizontal axis 17 is fastened onto the upper cross-beam 14 of the frame 12. In addition, the treatment head 7 can be moved along a vertical axis 4. The horizontal connection of the two side supports 13 of the frame 12, in the form of the upper cross-beam 14, pivots about angles 32 opposite to the pivot angle 33 when the upper part of the side supports 13 are deflected out about articulations 15. In this connection, the treatment head 7 remains in its vertical orientation.

To better reach the side skin regions of a patient, this coupling of the pivot movements can also be canceled out.

The controller 8 of the apparatus 11 is connected with the apparatus 11 by means of a rod holder 25. The controller 8 is alternatively connected with the power electronics of the various positioning drives of the treatment head 4 in cable-connected or radio-connected manner. Optionally, electrical spindle/nut drives or electrical linear drives are used as positioning drives.

FIG. 3 shows a view of a person 2 lying on the table 16. The body surface 6 that can be reached by the treatment head 7 is made evident by a light grid 10 projected onto the skin surface 24. The light grid divides the body surface 6 up into surface sections 9. A sub-set of these surface sections also contains portions of the treatment surface 21, the edging of which is marked with 20. Only the group 5 of the surface sections 9 also contains the treatment surface 21. Consequently, only this group is also approached by the treatment head.

### REFERENCE SYMBOL LIST

1.

2. person

3.

4.

5. group of surface sections

6. body surface

7. treatment head

8. controller

9. surface section

10. light grid

11. apparatus

12. frame

13. side supports

14. upper cross-beam

15. articulation

16. table

17. linear axis

18. adjustment drive

19.

20. edging

21. treatment surface

22.

23. pixel(s)

24. skin surface

25.

26. rod holder

27. articulation axis

28. starting surface

29. ending surface

30. sequence

31. stopping point

32. angle

33. angle

1. Method for irradiation or treatment of body surfaces with electromagnetic irradiation from a radiation source, wherein the body surface contains at least one treatment surface with irregular edges, which surface is determined and irradiated, wherein the body surface is divided up into a number of surface sections, which contain the treatment surface, at least in part, and wherein a treatment surface portion contained in each surface section is exposed to light with a radiation dose sequentially or in scrolling or step-by-step or targeted manner.

2. Method according to claim 1, wherein a topology of the treatment surface is determined.

3. Method according to claim 1, wherein a radiation dose for treatment of the treatment surface is determined.

4. Method according to claim 1, wherein the treatment surface is repeatedly determined.

5. Method according to claim 1, wherein a maximal radiation power distribution on the surface section and/or body surface is determined.

6. Method according to claim 1, wherein the one radiation power distribution is adapted to the local radiation dose distribution by means of modulation of a light modulator, preferably a micromirror actuator.

7. Method according to claim 1, wherein the radiation power density is adjusted by means of a change in an imaging scale of the light modulator, and wherein an image of the light modulator on the body surface is selected as a surface section.

8. Apparatus for irradiation or treatment of surfaces, comprising
at least one radiation source,
at least one treatment head having optics for imaging a light modulator on a body surface,
means for recognition of at least one treatment surface in the body surface to be irradiated,
at least one light modulator, particularly a micromirror actuator,
wherein the apparatus has a controller that is configured to divide the body surface up into surface sections, and has a position drive controlled by it, which drive is configured to direct the treatment head at the surface section as a function of the surface section to be irradiated.

9. Apparatus according to claim 8, wherein the apparatus has a table for a body to lie on and a portal for movable fastening of the treatment head.

10. Apparatus according to claim 9, wherein the portal is configured to be displaceable relative to the table.
